(19) United States
(12) Patent Application Publication
(10) Pub. No.: US 2025/0068603 A1
(43) Pub. Date: Feb. 27, 2025

(54) ELECTRONIC DEVICE AND OPERATING METHOD THEREOF FOR BUILDING FORMULATION DATABASE BASED ON ARTIFICIAL INTELLIGENCE

(71) Applicant: Heerae Co., Ltd., Seoul (KR)

(72) Inventors: Kyudam CHOI, Seoul (KR); Dooil KIM, Seongnam-si (KR); Taejun LEE, Gwangju-si (KR); Cheongwon KIM, Seoul (KR); Seongjin KIM, Seongnam-si (KR)

(73) Assignee: Heerae Co., Ltd., Seoul (KR)

(21) Appl. No.: 18/812,439

(22) Filed: Aug. 22, 2024

(30) Foreign Application Priority Data
Aug. 23, 2023 (KR) 10-2023-0110403
Feb. 8, 2024 (KR) 10-2024-0019940

(51) Int. Cl. G06F 16/21; G06N 20/00

(52) U.S. Cl. CPC G06F 16/212; G06N 20/00

(57) ABSTRACT

According to the present disclosure, the present disclosure provides an electronic device for building an artificial intelligence-based formulation database and an operating method thereof, the method including: extracting data from a document to build a formulation database, generating composite formulation data based on the formulation database, predicting, using a formulation property prediction model that infers a property change according to a formulation ratio, the property of a compound from the formulation information of the compound based on the composite formulation data, and optimizing a formulation using a formulation optimization model that generates a new compound formulation suitable for a target property in a compound formulation.

### CROSS-REFERENCE TO RELATED APPLICATIONS

The present application claims the benefit of priority to Korean Patent Application No. 10-2024-0037302, filed on Mar. 18, 2024, in the Korean Intellectual Property Office. The disclosures of the above-listed application are hereby incorporated by reference herein in their entirety.

### BACKGROUND OF THE INVENTION

### Field of the Invention

Embodiments of the present disclosure relate to an electronic device for building an artificial intelligence-based formulation database and an operating method thereof.

### Background of the Related Art

Recently, as investment in research and development has expanded and various technologies have developed rapidly, documents containing a large amount of information are being produced at a very rapid rate. For example, a number of research papers in the field of science and technology, a number of documents on patient case studies in the field of medicine, and a number of related legal documents or patent applications are increasing rapidly as legal conflicts increase.

In such an environment, it is difficult for individuals to read and analyze numerous documents on their own. In order to solve the foregoing difficulties, natural language processing technology utilizing artificial intelligence is attracting attention. Natural language processing technology utilizing artificial intelligence may provide meaningful information to people by analyzing text in documents. For example, a natural language processing device utilizing artificial intelligence may analyze a long piece of text and summarize its key points, or retrieve information of interest and recommend it to an individual. Through natural language processing utilizing artificial intelligence, people may receive the documents they need and information therein without a hassle. Furthermore, for natural language processing utilizing artificial intelligence, it is necessary to first extract text from electronic documents.

### SUMMARY OF THE INVENTION

However, in an electronic device in the related art as described and an operating method thereof, there is a problem in that data (e.g., formulation data and/or property data) needed by a user cannot be accurately extracted and processed from source data.

Embodiments of the present disclosure are intended to solve various problems including the foregoing problems, and aim to provide an electronic device for building an artificial intelligence-based formulation database that can accurately extract and process data needed by the user from source data, and an operating method thereof. However, these are illustrative, and the scope of the present disclosure is not limited thereto.

According to an aspect of the present disclosure, there is provided an operating method, performed by one or more processors of an electronic device, the operating method including extracting data from a document to build a formulation database, generating composite formulation data based on the formulation database, predicting, using a formulation property prediction model that infers a property change according to a formulation ratio, the property of a compound from the formulation information of the compound based on the composite formulation data, and optimizing a formulation using a formulation optimization model that generates a new compound formulation suitable for a target property in a compound formulation.

According to this embodiment, the building of the formulation database may include extracting a paragraph related to a formulation from text included in the document, and extracting data related to the formulation from a table included in the document.

According to this embodiment, the extracting of a paragraph related to the formulation may include extracting text segments in the document, predicting a class related to the formulation for the extracted text segments using an object name recognition model, and storing a paragraph including a sentence including information including at least one of the content and property of the formulation.

According to this embodiment, the extracting of data related to the formulation may include checking the locations of tables in the document using a pre-trained table-transformer model, setting a window of a preset size at the checked locations of the tables, extracting contents of a table included in the window, selecting a table including a content including a keyword related to the formulation from the extracted contents using a Tesseract OCR model, and storing the selected table.

According to this embodiment, the extracting of data related to the formulation may further include converting selected table images into an HTML format using a table structure recognition model, and converting and storing a table to fit the format of the existing database using an HTML parser.

According to this embodiment, the extracting of data related to the formulation may include checking a row having a composition ratio or a property in a table included in at least one of an HTML document and an XML document, sorting example numbers and composition information or property information in a table head, extracting data whose example numbers match from a composition data set and a property data set, storing information including the name, unit, and amount of a raw material, storing information including the name, value, unit, method, and condition of a property, and automatically adding composition ratio information and property information to an existing database.

According to this embodiment, the generating of the composite formulation data may include generating composite formulation data similar to actual formulation data from original data using a tabular data generator, comparing a conditional distribution between the original data and the composite formulation data similar to the actual formulation data using a discriminator to output a similarity, and sampling composite data having a distribution diagram similar to the original data based on the similarity.

According to this embodiment, the predicting of the property of the compound may include collecting learning data including preprocessed original data and the composite formulation data, selecting a property value to be predicted by the formulation property prediction model, and then defining the representation method of data, storing a weight value of a model for each property that has been trained through knowledge transfer and carrying out performance evaluation on untrained data, and evaluating a prediction result for each predicted property.

According to this embodiment, the optimizing of the formulation may include generating third data for a new compound formulation based on first data for a target property and second data for an existing compound formulation, assigning, in an interactive environment, reward score based on a similarity between a property value of the new compound formulation of the third data and the target property, and feeding back, in a critic network, an expected value of how close the new formulation changed compared to an existing formulation has improved to the target property based on the reward score to the actor network.

According to another aspect of the present disclosure, an electronic device may include a memory that stores one or more instructions, and a processor that executes the one or more instructions stored in the memory, wherein the processor may execute extracting data from a document to build a formulation database, generating composite formulation data based on the formulation database, predicting, using a formulation property prediction model that infers a property change according to a formulation ratio, the property of a compound from the formulation information of the compound based on the composite formulation data, and optimizing a formulation using a formulation optimization model that generates a new compound formulation suitable for a target property in a compound formulation.

Other aspects, features and advantages other than those described above will become apparent from the detailed description, claims and drawings for carrying out the disclosure below.

In addition, these general and specific aspects may be carried out by using a system, a method, a computer program, or a combination thereof.

According to an exemplary embodiment of the present disclosure configured as described above, an electronic device capable of building an artificial intelligence-based formulation database for designing a compound having properties desired by a user and an operating method thereof may be implemented. Of course, the scope of the present disclosure is not limited by this effect.

### BRIEF DESCRIPTION OF THE DRAWINGS

FIG. 1 is a block diagram schematically showing a system according to an exemplary embodiment of the present disclosure.

FIG. 2 is a block diagram schematically showing an electronic device according to an exemplary embodiment of the present disclosure.

FIG. 3 is a flowchart for explaining a method of operating the electronic device of FIG. 2.

FIG. 4 is a diagram for explaining a process of extracting text according to an exemplary embodiment of the present disclosure.

FIG. 5 is a diagram for explaining a process of extracting a table according to an exemplary embodiment of the present disclosure.

FIG. 6 is a diagram showing an example of a process of extracting a table according to an exemplary embodiment of the present disclosure.

FIG. 7 is a diagram for explaining a process of a model of recognizing a structure of a table according to an exemplary embodiment of the present disclosure.

FIG. 8 is a flowchart for explaining a process of a parser according to an exemplary embodiment of the present disclosure.

FIG. 9 is a diagram showing an example in which extraction forms of tables according to an exemplary embodiment of the present disclosure are compared with each other.

FIG. 10 is a diagram showing an example of finally stored composition and property tables according to an exemplary embodiment of the present disclosure.

FIGS. 11A to 11F are diagrams showing examples of types of table forms included in a patent document according to an exemplary embodiment of the present disclosure.

FIG. 12 is a diagram for explaining a process of generating composite formulation data according to an exemplary embodiment of the present disclosure.

FIG. 13 is a diagram showing an example of converting data of a property table according to an exemplary embodiment of the present disclosure.

FIG. 14 is a diagram showing an example of converting a property table according to an exemplary embodiment of the present disclosure.

FIG. 15 is a diagram showing an example of a frame of data that is input to a model according to an exemplary embodiment of the present disclosure.

FIG. 16 is a diagram illustratively showing distribution diagrams of composite formulation data generated by a model according to an exemplary embodiment of the present disclosure.

FIG. 17 is a diagram for explaining a formulation property prediction process according to an exemplary embodiment of the present disclosure.

FIG. 18 is a diagram showing examples of data generated by a formulation property prediction model according to an exemplary embodiment of the present disclosure.

FIG. 19 is a diagram for explaining a formulation optimization process according to an exemplary embodiment of the present disclosure.

### DETAILED DESCRIPTION OF THE PREFERRED EMBODIMENT

As various modifications can be made and diverse embodiments are applicable to the present disclosure, specific embodiments will be illustrated with reference to the accompanying drawings and described in detail through the following description. Effects and features of the present disclosure, and methods of accomplishing the same will be clearly understood with reference to the following embodiments described below in detail in conjunction with the accompanying drawings. However, the present disclosure is not limited to the embodiments which will be disclosed below, but may also be implemented in various forms.

In the present disclosure, the terms such as “first,” and “second” are used not in a limiting sense but for the purpose of distinguishing one element from another element. Therefore, a first element mentioned below may, of course, be a second element within the technical concept of the present disclosure.

In the present disclosure, a singular expression may include a plural expression unless they are definitely different in a context. In addition, the phrases “A and/or B” and “at least one of A and B” denote A, B or A and B.

In the present disclosure, an expression such as “include” or “have” denote the presence of features and/or elements described herein, but do not preclude the presence or addition of one or more other features and/or elements thereof.

In the present disclosure, the expression “exemplary” is used to refer to “being used as an example or illustration.” In the present disclosure, any embodiment described as “exemplary” should not necessarily be construed as preferred or as having an advantage over other embodiments.

In the drawings herein, elements may be exaggerated or reduced in size for convenience of explanation. For instance, a size and thickness of each element shown in the drawings are arbitrarily shown for convenience of explanation, and thus the present disclosure is not necessarily limited thereto.

When an embodiment can be implemented differently, a specific operation sequence may be performed differently from the described sequence. For example, two consecutively described steps may be performed substantially at the same time, or may be performed in a sequence opposite to the described sequence.

Embodiments of the present disclosure may be described in terms of a function or a block that performs a function. A block referred to as a “unit” or a “module” herein may be physically implemented by an analog or digital circuit such as a logic gate, an integrated circuit, a microprocessor, a microcontroller, a memory, a passive electronic component, an active electronic component, an optical component, a hardwired circuit, and the like, and may optionally be driven by firmware and software. In addition, the term “unit” as used herein refers to software or a hardware element such as an FPGA, or an ASIC, and the “unit” may perform certain roles. However, the term “unit” is not limited to software or hardware. The term “unit” may be configured in a recording medium that can be addressed or may be configured to be reproduced on at least one processor. Therefore, as an example, the term “unit” may include elements such as software elements, object-oriented software elements, class elements, and task elements, processes, functions, properties, procedures, subroutines, segments in program codes, drivers, firmware, microcodes, circuits, data, databases, data structures, tables, arrays, and variables. Functions provided in the elements and the “units” may be combined as a smaller number of elements and “units” or further divided into additional elements and “units.”

Embodiments of the present disclosure may be implemented by using at least one software program running on at least one hardware device and may perform network management functions to control elements.

Unless otherwise defined, all terms (including technical and scientific terms) as used herein may be used with meanings that can be commonly understood by those skilled in the art to which the present disclosure pertains.

Hereinafter, embodiments of the present disclosure will be described in detail with reference to the accompanying drawings, and when the description is given with reference to the drawings, the same or corresponding elements are designated with the same numeral references and a redundant description thereof will be omitted.

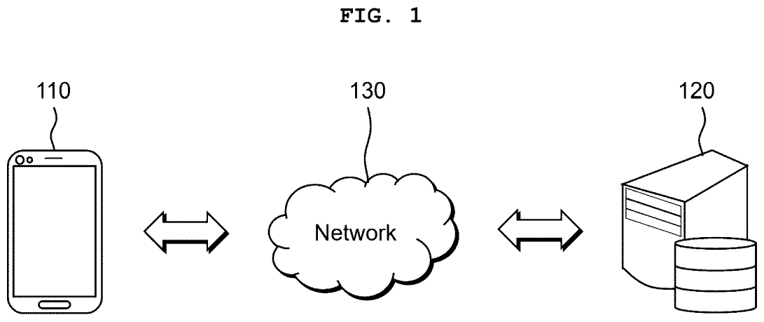

FIG. 1 is a block diagram schematically showing a system 100 according to an exemplary embodiment of the present disclosure.

Referring to FIG. 1, the system 100 may include a user terminal 110, an electronic device 120, and a network 130. The components (or elements) shown in FIG. 1 are illustrative, and additional components may be present or some of the components shown in FIG. 1 may be omitted. The user terminal 110 and the electronic device 120 in the present disclosure may transmit and receive data to and from each other for the system according to exemplary embodiments of the present disclosure through the network 130.

The user terminal 110 may transmit various data to the electronic device 120. The user terminal 110 may refer to any type(s) of entity (entities) in the system having a mechanism for communication with the electronic device 120. The user terminal 110 is a device capable of communication and there is no limitation to its form. For instance, the user terminal 110 according to the present disclosure may be in the form of any one or a combination of two or more of a computer, a server device, and a portable terminal.

The electronic device 120 may communicate with the user terminal 110, and execute a program of program data. In this specification, a ‘device according to the present disclosure’ may include all various devices that can perform computational processing and provide a result thereof to a user. For instance, the electronic device 120 according to the present disclosure, which is a computing device, may be in the form of any one or a combination of two or more of a computer, a server device, and a portable terminal.

The network 130 may be configured regardless of its communication mode, such as wired or wireless. For instance, the network 130 may be configured with various communication networks, such as a personal area network (PAN), a short-distance network (WAPN), a local area network (LAN), and a wide area network (WAN).

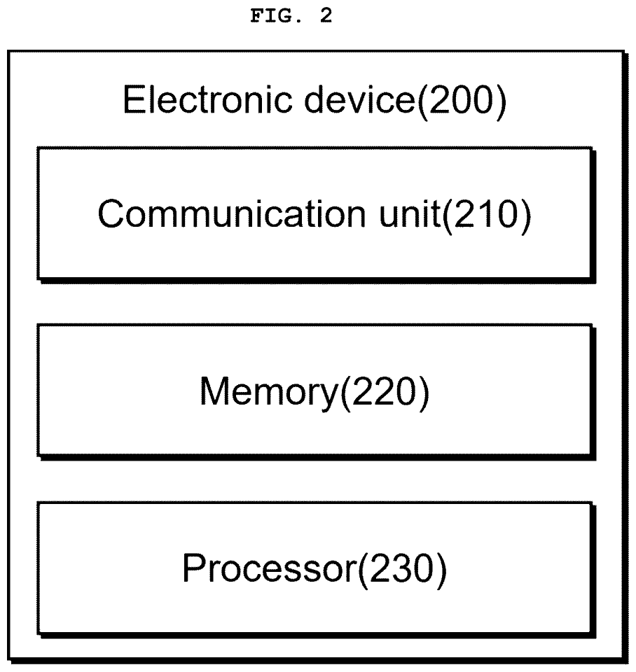

FIG. 2 is a block diagram schematically showing an electronic device 200 according to an exemplary embodiment of the present disclosure.

Referring to FIG. 2, the electronic device 120, may include a communication unit 210, a memory 220, and a processor 230.

The communication unit 210 may communicate with at least one user terminal 110.

The memory 220 may store data for an algorithm for controlling the operation of elements in the electronic device 120 of the present disclosure or a program that reproduces the algorithm, and may be implemented with at least one processor 230 that performs the foregoing operation using the data stored in the memory 220. Here, the memory 220 and the processor 230 may each be implemented as a separate chip. Furthermore, the memory 220 and the processor 230 may be implemented as a single chip.

The memory 220 may store data supporting various functions of the electronic device 120 of the present disclosure and a program for operating the processor 230. The memory 220 may store input/output data, and store a plurality of application programs (or applications) running on the device, data items, commands, and one or more instructions for the operation of the device. In one embodiment, at least some of the application programs may be downloaded from an external server via wireless communication.

The memory 220 may include at least one type of storage medium including a Flash memory, a hard disk, a multimedia card micro type, a card-type memory (e.g., SD or DX memory, etc.), a Random Access Memory (RAM), a Static Random Access Memory (SRAM), a Read-Only Memory (ROM), an Electrically Erasable Programmable Read-Only Memory (EEPROM), a Programmable Read-Only memory (PROM), a magnetic memory, a magnetic disk, and an optical disk, but is not limited thereto. Furthermore, the memory 220 is separate from the device, but may be a database connected by wire or wirelessly.

The processor 230 may execute the one or more instructions stored in the memory 220. The processor 230 may extract data from a document to build a formulation database, generate composite formulation data based on the formulation database, predict, using a formulation property prediction model that infers a property change according to a formulation ratio, the property of a compound from the formulation information of the compound based on the composite formulation data, and optimize a formulation using a formulation optimization model that generates a new compound formulation suitable for a target property in a compound formulation.

A function related to artificial intelligence according to the present disclosure may be operated through the processor 230 and the memory 220. The processor 230 may be configured with one or more processors. Here, the one or more processors may be a general-purpose processor such as a CPU, an AP, and a digital signal processor (DSP), a graphics dedicated processor such as a graphic processing unit (GPU), a vision processing unit (VPU), or an artificial intelligence dedicated processor such as a neural processing unit (NPU). The one or more processors may be controlled to process input data according to a predefined operation rule or artificial intelligence model stored in the memory 220. Alternatively, when the one or more processors are dedicated artificial intelligence processors, they may be designed with a hardware structure specialized for processing a specific artificial intelligence model.

The predefined operation rule or artificial intelligence model may be characterized by being created through learning. Here, being created through learning denotes that a basic artificial intelligence model is trained using a large number of learning data items by a learning algorithm so as to create a predefined operation rule or artificial intelligence model set to perform a desired characteristic (or purpose). The learning may be carried out in an apparatus itself that performs artificial intelligence according to the present disclosure, or may be carried out through a separate server and/or a system. Examples of learning algorithms include supervised learning, unsupervised learning, semi-supervised learning, or reinforcement learning, but are not limited to the above-described examples.

An artificial intelligence model may be configured with a plurality of neural network layers. Each of the plurality of neural network layers has a plurality of weight values, and a neural network calculation may be performed through a calculation between a calculation result of a previous layer and the plurality of weight values. The weight values of the plurality of neural network layers may be optimized by a learning result of the artificial intelligence model. For example, a plurality of weight values may be updated such that a loss value or cost value acquired from the artificial intelligence model is reduced or minimized during the learning process. The artificial neural network may include a deep neural network (DNN), for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), a deep Q-network, or the like, but is not limited to the above-described examples.

The processor 230 may generate a neural network, train or learn a neural network, or perform a calculation based on received input data, and generate an information signal based on the execution result or retrain the neural network. Models of neural networks may include a convolution neural network (CNN), such as GoogleNet, AlexNet, a VGG network, a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) network, a classification network, and the like, but are not limited thereto. The processor 230 may include one or more processors to perform calculations according to the models of neural networks. For example, the neural network may include a deep neural network.

The neural network may include a convolutional neural network (CNN), a recurrent neural network (RNN), a perceptron, a multilayer perceptron, a feed forward (FF), a radial basis function network (RBFN), a deep feed forward (DFF), a long short term memory (LSTM), a gated recurrent unit (GRU), an auto encoder (AE), a variational auto encoder (VAE), a denoising auto encoder (DAE), a sparse auto encoder (SAE), a Markov chain (MC), a Hopfield network (HN), a Boltzmann machine (BM), a restricted Boltzmann machine (RBM), a deep belief network (DBN), a deep convolutional network (DCN), a deconvolutional network (DN), a deep convolutional inverse graphics network (DCIGN), a generative adversarial network (GAN), a liquid state machine (LSM), an extreme learning machine (ELM), an echo state network (ESN), a deep residual network (DRN), a differentiable neural computer (DNC), a neural turning machine (NTM), a capsule network (CN), a Kohonen network (KN), and an attention network (AN), but it will be understood by those skilled in the art that the neural network is not limited thereto and may include any neural network.

According to an exemplary embodiment of the present disclosure, the processor 230 may use various artificial intelligence structures and algorithms, such as a convolution neural network, such as GoogleNet, AlexNet, or a VGG network, a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBN), a fully convolutional network, a long short term memory (LSTM) network, a classification network, generative modeling, explainable AI, continual AI, representation learning, AI for material design, BERT for processing natural languages, SP-BERT, MRC/QA, a text analysis, a dialog system, GPT-3, GPT-4, visual analytics for processing visions, visual understanding, video synthesis, anomaly detection for ResNet data intelligence, prediction, time-series forecasting, optimization, recommendation, data creation, and the like, but the present disclosure is not limited thereto.

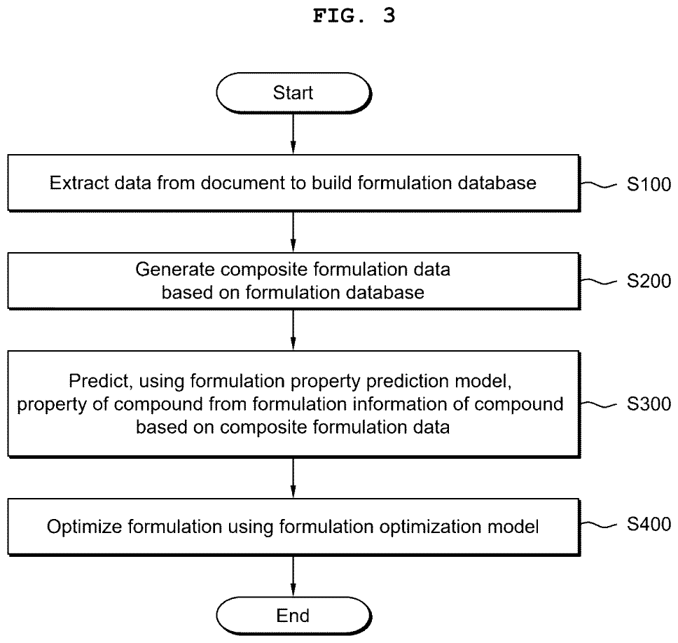

FIG. 3 is a flowchart for explaining a method of operating the electronic device 200 of FIG. 2. The operation method in FIG. 3 may be performed by one or more processors 230 of the electronic device 200. Part of the operating method in FIG. 3 may be omitted or an order thereof may also be changed.

Referring to FIG. 3, an operating method according to an exemplary embodiment of the present disclosure may include extracting data from a document to build a formulation database (S100), generating composite formulation data based on the formulation database (S200), predicting, using a formulation property prediction model that infers a property change according to a formulation ratio, the property of a compound from the formulation information of the compound based on the composite formulation data (S300), and optimizing a formulation using a formulation optimization model that generates a new compound formulation suitable for a target property in a compound formulation (S400). A detailed description of the steps S100 to S400 will be described later.

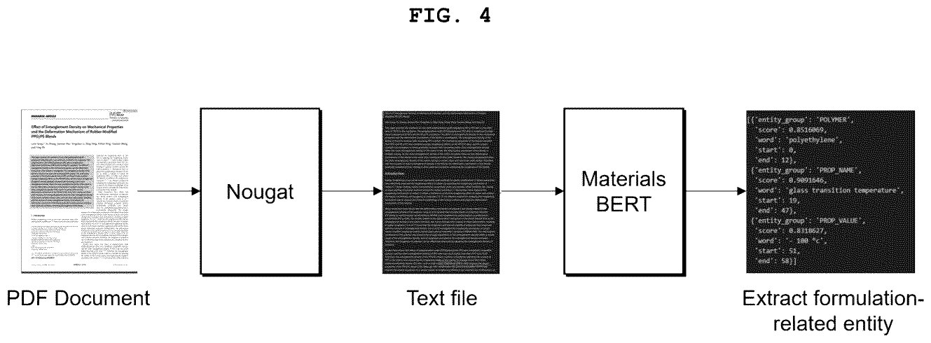

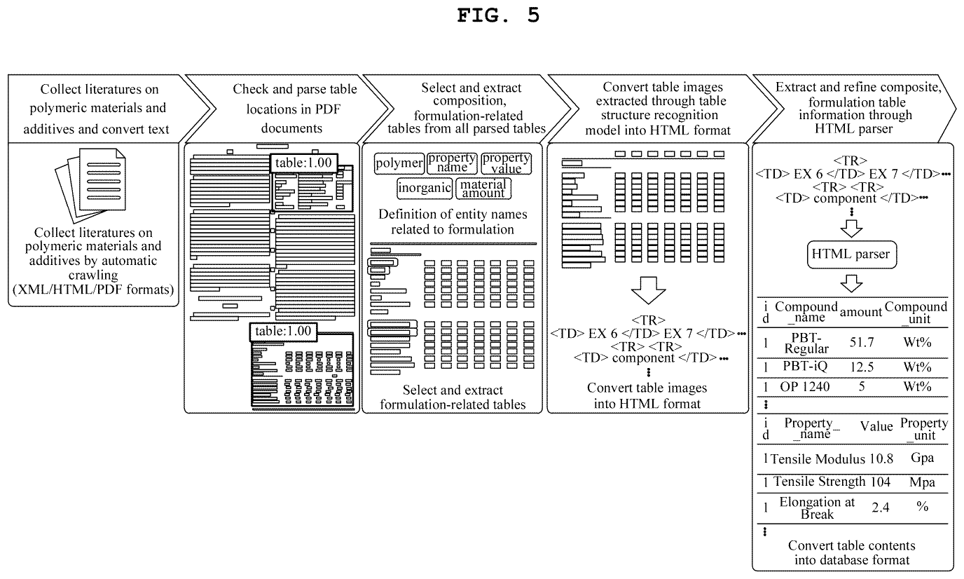

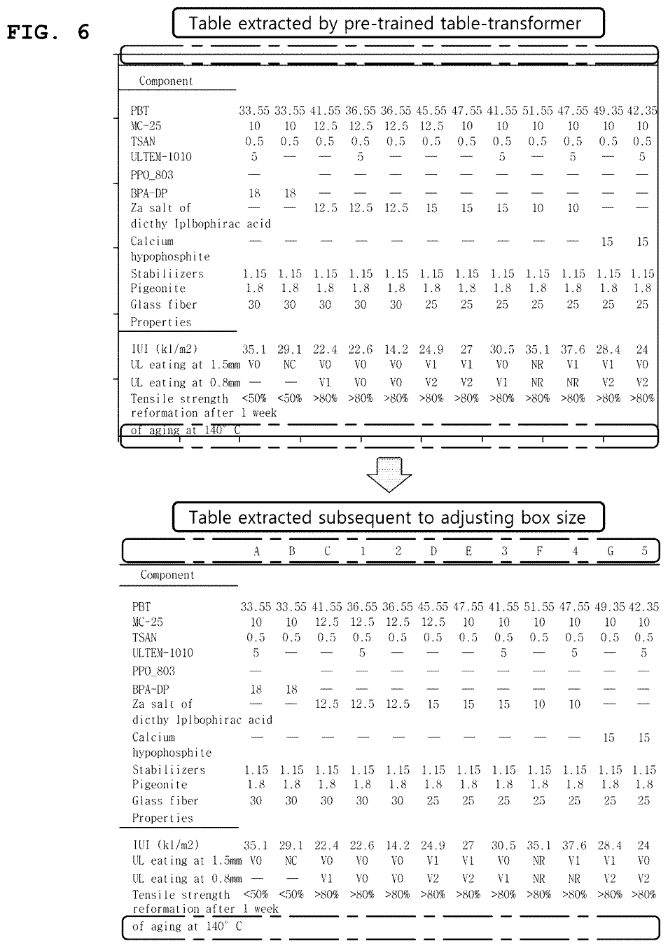

FIG. 4 is a diagram for explaining a process of extracting text according to an exemplary embodiment of the present disclosure, FIG. 5 is a diagram for explaining a process of extracting a table according to an exemplary embodiment of the present disclosure, and FIG. 6 is a diagram showing an example of a process of extracting a table according to an exemplary embodiment of the present disclosure.

Referring to FIGS. 3 to 6, a formulation database may be a database built by securing data items for a formulation and property values from source data. Here, the source data may be data including at least one of text, tables, and images related to the formulation and property values. In one embodiment, source data may be document data such as a patent document or paper, and may be a document in a PDF format, but is not limited thereto. Hereinafter, a case where the source data is document data will be described as an example.

In one embodiment, in step S100, the one or more processors 230 may include extracting a paragraph related to a formulation from text included in a document, and extracting data related to the formulation from a table (or a chart) included in the document.

Referring to FIG. 4, in an exemplary embodiment, the extracting of a paragraph related to the formulation may include extracting text segments in the document, predicting a class related to the formulation in a sentence that is input with the extracted text segments using an object name recognition model, and storing a paragraph including a sentence including information including at least one of the content and property of the formulation.

In one embodiment, in the extracting of text segments in a document, the processor 230 may extract text segments included in a document in a PDF format (hereinafter, referred to as a “PDF document”). In an exemplary embodiment, the processor 230 may extract text segments included in a PDF document by generating (or converting) a file related to software for molecular modeling of biopolymer organic compounds.

In an exemplary embodiment, a model for extracting text may be implemented as an optical character recognition (OCR)-based model (e.g., Nougat) to recognize a program such as Python. In addition, a file related to software may be implemented as an mmd file. However, the present disclosure is not limited to the above-described embodiment.

In one embodiment, in the predicting of a class related to the formulation, the processor 230 may predict, using an object name recognition model, a class (or entity) related to a formulation in a sentence that is input to the object name recognition model for the extracted text. When there is a sentence including content information or property information of a formulation, the processor 230 may store the corresponding paragraph in a database, or the like. For instance, the object name recognition model may be implemented with MaterialsBERT, but is not limited thereto.

Referring to FIG. 5, in an exemplary embodiment, the extracting of data related to the formulation may include checking the locations of tables in the document using a pre-trained model that extracts a table, setting a window of a preset size at the checked locations of the tables, extracting contents of a table included in the window, selecting a table including a content including a keyword related to the formulation from the extracted contents using a Tesseract OCR model, and storing the selected table.

In an exemplary embodiment, the processor 230 may collect literatures on polymeric materials and additives, and convert text in the literature. For instance, the collection method may collect literatures using an automatic crawling method and convert text included in the collected literatures into documents in formats such as XML, HTML, and PDF.

In addition, in the checking of the locations of tables in a document, the processor 230 may check the locations of tables in a PDF document using a pre-trained table-transformer model, and then separately extract only tables in the PDF document.

In one embodiment, when extracting a table, the processor 230 may designate a window (e.g., a box-shaped region of interest) corresponding to a size of the table and extract the contents of the table at the location of the window in the PDF document.

Meanwhile, in a process of extracting the contents of the table, at least part of a region adjacent to an edge of the table may be cut off. In order to prevent this, the processor 230 may additionally adjust the size of the window to extract an entire table including all the contents of each table.

As a specific example, the processor 230 may expand the size of the window such that the window is spaced from the edge of the table (e.g., the border of the table, such as a top, a bottom, a left, and a right in the table), and includes a preset margin. Here, a size of the margin may be 10 mm to 15 mm, preferably 11 mm to 13 mm, and more preferably 11.5 mm to 12.5 mm, but is not limited thereto and may be variously modified depending on the setting.

In an exemplary embodiment, the extracting of data related to the formulation may further includes converting selected table images into an HTML format using a table structure recognition model, and converting and storing a table to fit the format of the existing database using an HTML parser. For instance, the processor 230 may parse a table whose location has been confirmed, select and extract a table related to composition and formulation from the parsed table, convert an image for the extracted table into an HTML format through a table structure recognition model, and convert and refine information related to composition and formulation in the table into a database format through an HTML parser.

Referring to FIGS. 5 and 6, a table shown at the top of FIG. 6 may be an example of a table extracted in a relatively small-sized window (e.g., box), in which first and last rows of the table are excluded, and a table shown at the bottom of FIG. 6 may be an example of a table extracted after a size of the window (e.g., a box size) is adjusted, in which an entire table including all of the contents of the table is extracted. Specifically, referring to FIG. 6, it is shown an example in which a table initially extracted (a table shown at the top of FIG. 6) has first and last lines truncated, but after adjusting a size of the box, an entire table (a table shown at the bottom of FIG. 6) is extracted.

In one embodiment, when an entire table is extracted, the processor 230 may recognize the contents of the table using a table content recognition model. In addition, the table content recognition model may be implemented as a Tesseract OCR model, but the present disclosure is not limited to the embodiment. In an exemplary embodiment, the processor 230 may recognize the contents of the table using the Tesseract OCR model, check whether a keyword related to the formulation is included, and select only tables related to the formulation. The processor 230 may convert the selected table images into an HTML format using a table structure recognition model. Then, the processor 230 may convert the table converted into the HTML format to fit the format of the existing database using an HTML parser and store the contents of the table converted to the database format.

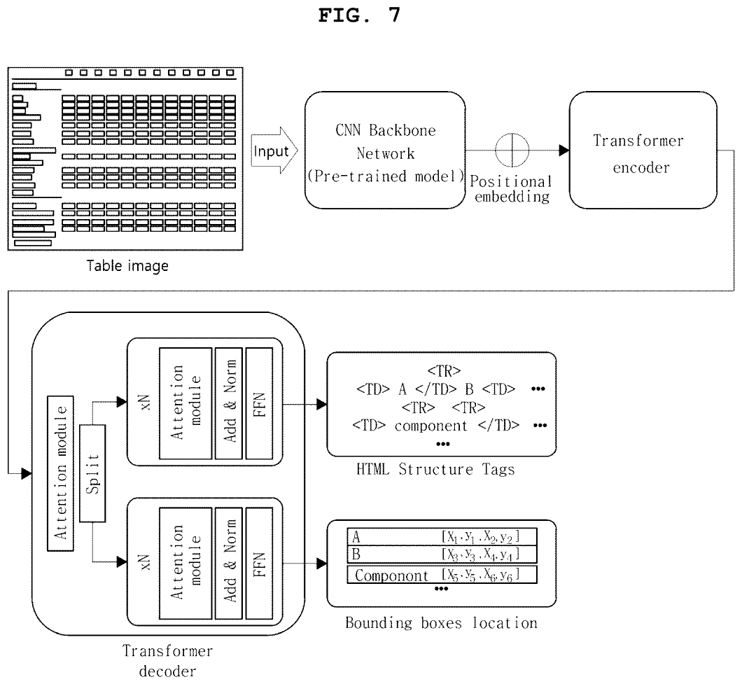

FIG. 7 is a diagram for explaining a process of a model of recognizing a structure of a table according to an exemplary embodiment of the present disclosure.

Referring to FIG. 7, a table image may be input to a pre-trained model. In an exemplary embodiment, the pre-trained model may be implemented as a CNN backbone network, but is not limited thereto. The output of a pre-trained model may be input to a transformer. For instance, the output of a CNN backbone network may be positionally embedded in a transformer encoder. The output of the transformer encoder may be input to a transformer decoder. The transformer decoder included in the model of the present disclosure may be configured with an attention module and two independent branches.

In one embodiment, a first branch may be configured to predict a structure, and a second branch may be configured to predict a bounding-box (bbox). Since the first and second branches do not share weight values, each branch may be trained to be optimized for a task to be predicted. Two tasks that are related to each other may partially cooperate with each other by sharing an attention module prior to being input to each branch. Each branch may include an attention module, add & norm, and a feed forward network (FFN) or feed forward neural network (FFNN). The output of the transformer decoder may include HTML structure tags and bounding boxes location. The model of the present disclosure may improve the accuracy of each region of a bounding-box (bbox) of cells so as to have no dividing lines and contain a lot of white space, thereby improving recognition rate when converting into a data frame even in a table with many empty regions on the image.

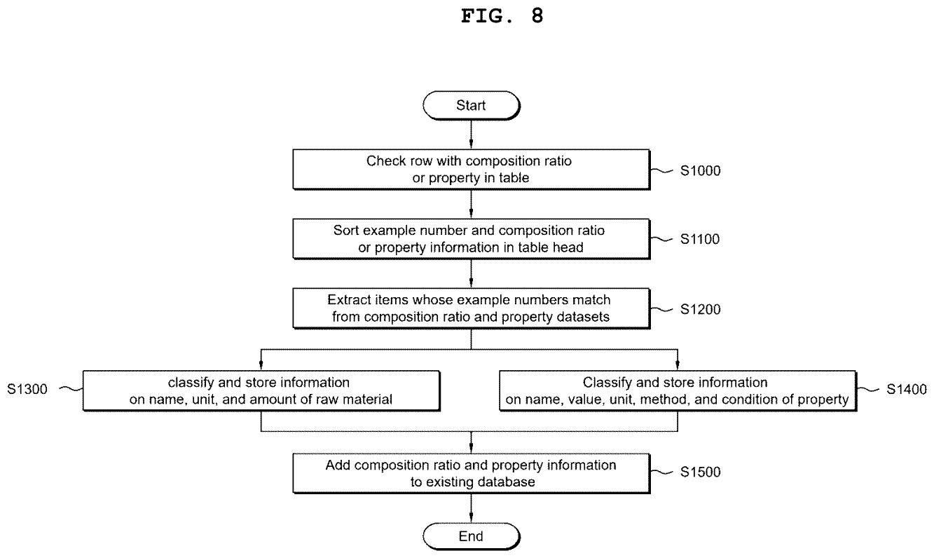

FIG. 8 is a flowchart for explaining a process of a parser according to an exemplary embodiment of the present disclosure.

Referring to FIG. 8, the processor 230 checks a row in a table containing a composition ratio or property (S1000). The processor 230 sorts an example number and composition ratio information or property information in a table head (S1100). The processor 230 extracts only those whose example numbers match from the composition ratio and property datasets (S1200). The processor 230 classifies and stores information on the name, unit, and amount of a raw material (S1300). In addition, the processor 230 classifies and stores property name, value, unit, method, and condition information (S1400). Next, the processor 230 automatically adds composition ratio and property information to an existing database (S1500). That is, when a document is HTML or XML, a parser of the present disclosure may identify various table forms in an HTML document or XML document, thereby extracting and refining composition ratio and property information as a formal table without being affected by a table form.

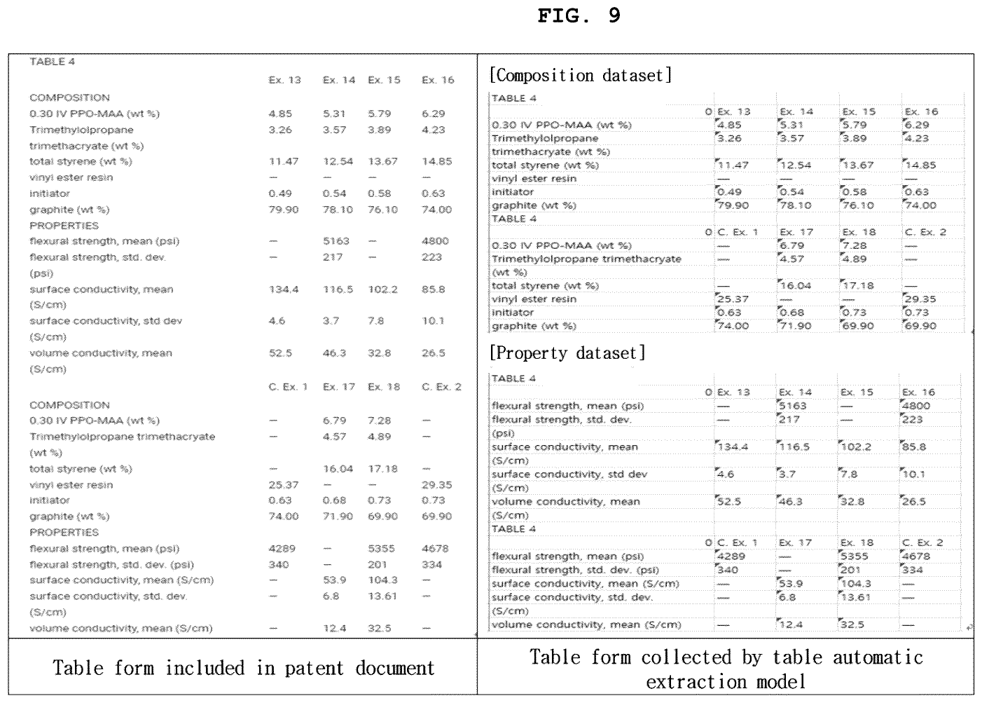

FIG. 9 is a diagram showing an example in which extraction forms of tables according to an exemplary embodiment of the present disclosure are compared with each other.

Referring to FIG. 9, for example, when the parser of the present disclosure is applied to the Google patent site, it is assumed that a user specifies the search words as “(ppo) and (tensile strength) and (formulation)) and (compounding)”, the material keyword as “ppo”, and the property keyword as “flexural strength”. In the model of the present disclosure, when there is no table containing the word “ppo” in patent documents obtained by entering a search word in Google Patent, the corresponding patent may be skipped. For instance, it is assumed that a table included in a patent document retrieved from Google Patent is as shown at the left side of FIG. 9. If the table includes material and/or property keywords, then the model of the present disclosure may extract the table as it is. The extracted composition table and property table may be stored in the composition dataset and property dataset as shown at the right side of FIG. 9. However, the present disclosure is not limited to the example shown in FIG. 9.

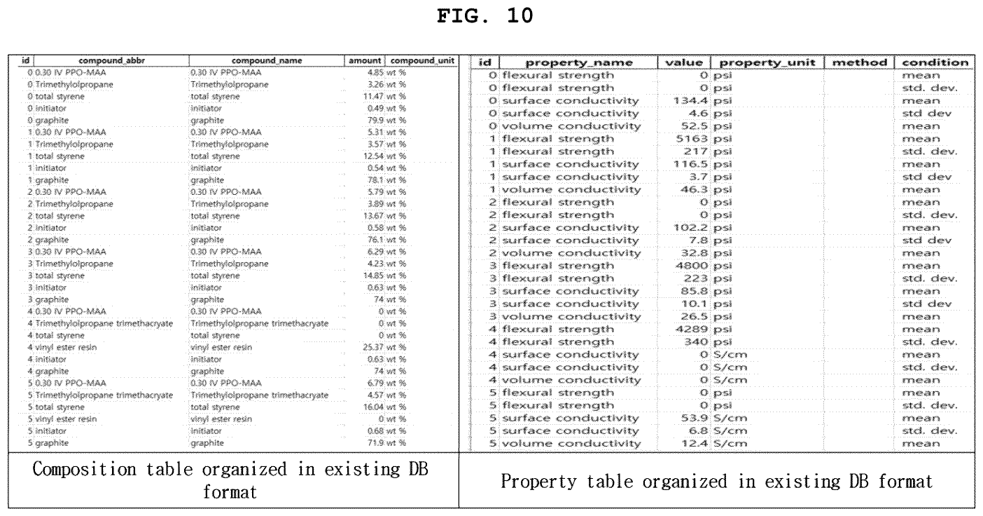

FIG. 10 is a diagram showing an example of finally stored composition and property tables according to an exemplary embodiment of the present disclosure.

Referring to FIG. 10, a primary composition data set and/or a primary property data set stored in a table form as it is in a document may be input to a database automatic addition model. The automatic database addition model may select only composition and/or property pairs with matching example numbers (Ex. 13, etc.) from a composition table and/or a property table and add them to an existing database. Here, additional information items displayed on a table, such as units, property experimental methods, and property experimental conditions, may be classified and automatically added to the existing database. Accordingly, a composition database and/or a property database as shown in FIG. 10 may be built. However, the present disclosure is not limited to the example shown in FIG. 10.

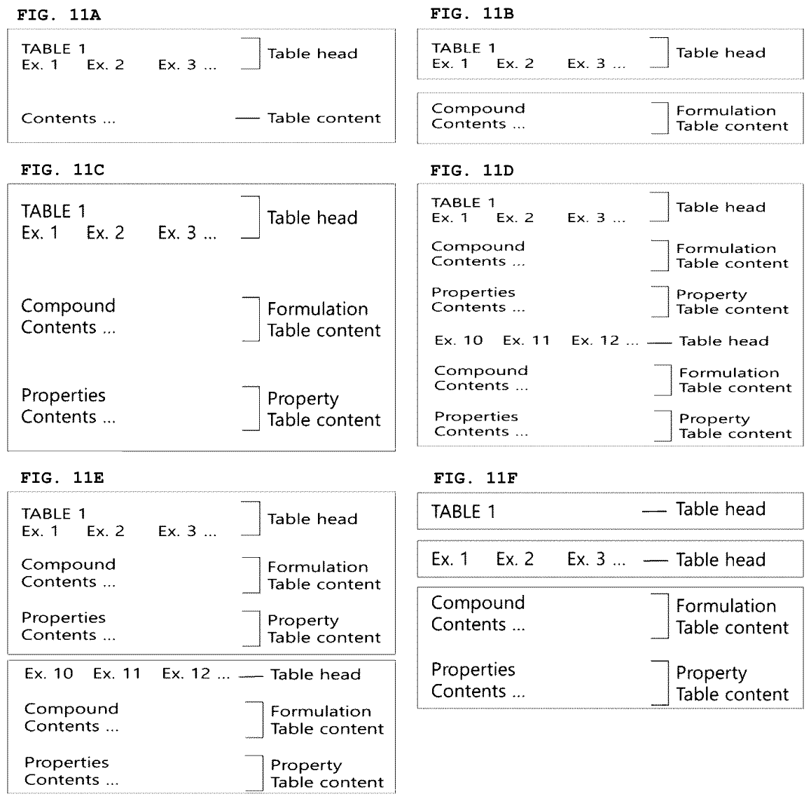

FIGS. 11A to 11F are diagrams showing examples of types of table forms included in a patent document according to an exemplary embodiment of the present disclosure.

Table (or chart) data included in a patent document may be classified into a plurality of cases depending on its form. The processor 230 may determine which of a plurality of preset classifications corresponds to table data in a patent document, and apply a different data extraction method to improve the accuracy of the data extraction process.

In one embodiment, as shown in FIGS. 11A to 11F, table data included in a patent document may be classified into six cases. In addition, the table data may include a table head and a table content, and the table content may include at least one of a formulation table content and a property table content.

In an exemplary embodiment, cases of table data may include six cases shown in FIGS. 11A to 11F. Specifically, the cases of table data may include a case where table heads and table contents are included in a single table (see FIG. 11A), and a case where at least part of table heads and table contents are included in different tables (see FIG. 11B), a case where table heads and table contents (e.g., composition data and property data) are all included in a single table (see FIG. 11C), a case where table heads and two or more table contents (e.g., composition data and property data pairs) are all included in a single table (see FIG. 11D), a case where table heads and one or more table contents (e.g., composition data and property data pairs) are included in a single table, but the table heads and the table contents (e.g., composition data and property data pairs) connected to one another are included in different, separate tables (see FIG. 11E), and a case where table heads are divided into two or more tables and included in different tables, and table contents (e.g., composition data and property data pairs) are included in another table (see FIG. 11F).

However, the present disclosure is not limited to the foregoing example, and case classification criteria and a number thereof may be modified in various ways.

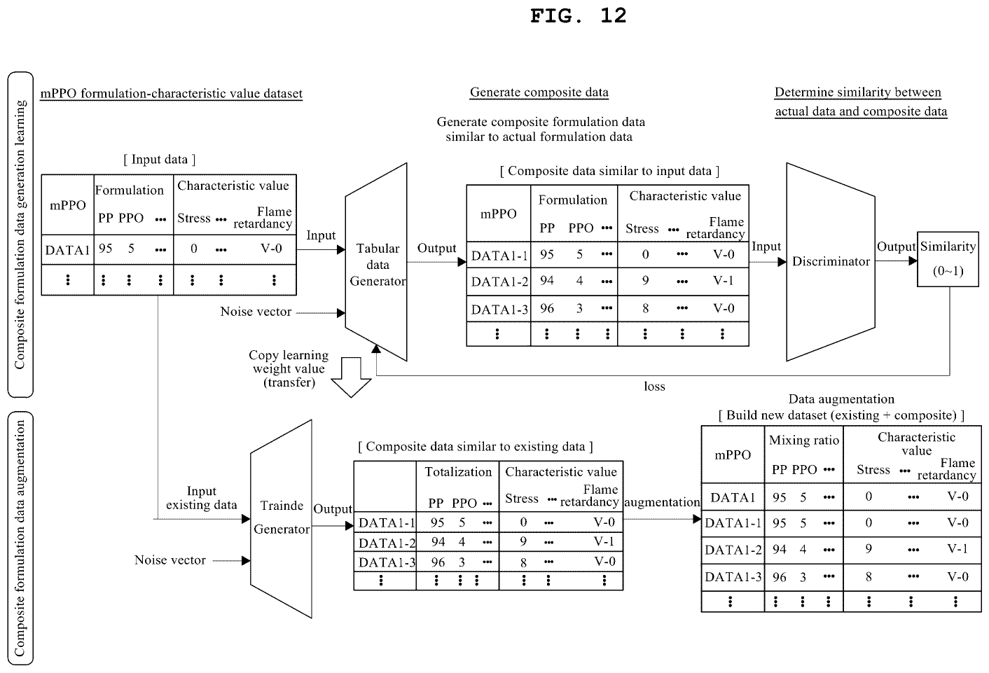

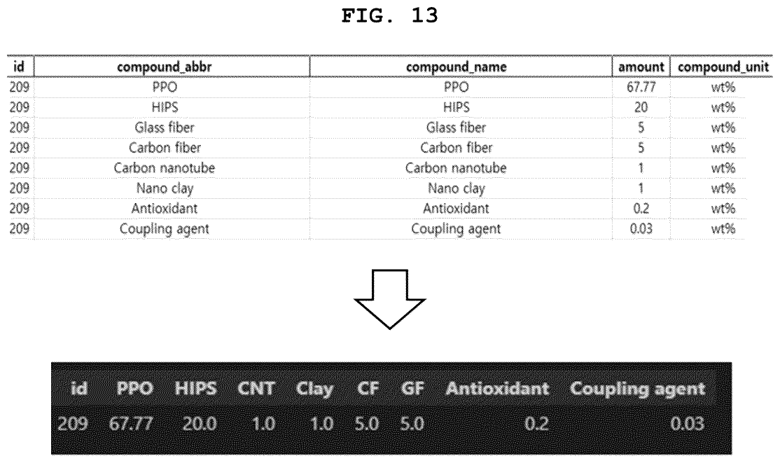

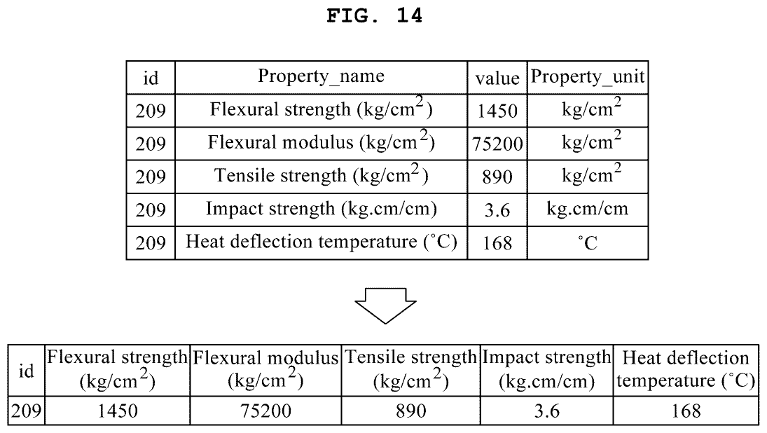

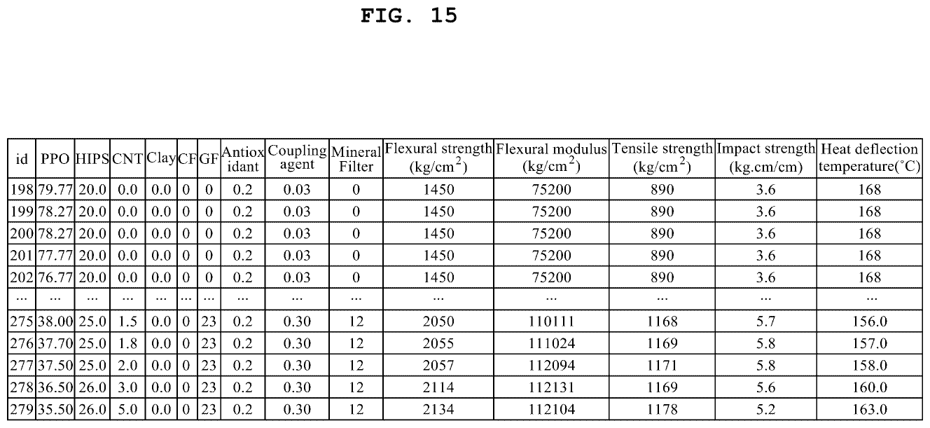

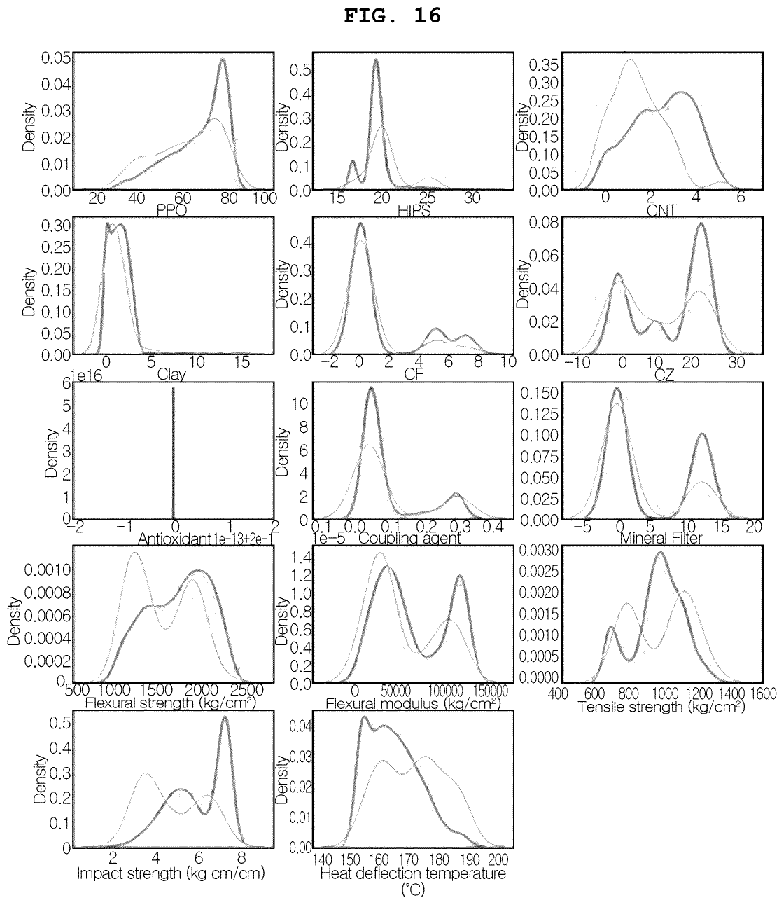

FIG. 12 is a diagram for explaining a process of generating composite formulation data according to an exemplary embodiment of the present disclosure, FIG. 13 is a diagram showing an example of converting data of a property table according to an exemplary embodiment of the present disclosure, FIG. 14 is a diagram showing an example of converting a property table according to an exemplary embodiment of the present disclosure, FIG. 15 is a diagram showing an example of a frame of data that is input to a model according to an exemplary embodiment of the present disclosure, and FIG. 16 is a diagram illustratively showing distribution diagrams of composite formulation data generated by a model according to an exemplary embodiment of the present disclosure.

Referring to FIGS. 12 to 16, the generation of composite formulation data may be intended to overcome the corresponding limitation because it is difficult to acquire minimum data for learning a property prediction model due to a limit to the number of published experimental data.

In one embodiment, in step S200, the processor 230 may include generating composite formulation data similar to actual formulation data from original data using a tabular data generator, comparing a conditional distribution between the original data and the composite formulation data similar to the actual formulation data using a discriminator to output a similarity, and sampling composite data having a distribution diagram similar to the original data based on the similarity.

Referring to FIG. 12, in composite formulation data generation learning, mPPO formulation and characteristic value datasets may be prepared as input data. Input data may be input to a tabular data generator. In addition, a noise vector may be input to the tabular data generator.

In one embodiment, the tabular data generator may generate composite data based on the input data. The tabular data generator may sample composite data according to a log-frequency in each category of the original data to prevent data belonging to minority categories from being generated. In an exemplary embodiment, composite formulation data similar to actual formulation data may be generated. For example, composite data similar to input data may be generated as shown in FIG. 12.

In one embodiment, composite data may be input to a discriminator. Referring to FIG. 12, the discriminator may determine a similarity between actual data and composite data. In an example embodiment, the discriminator may calculate a similarity by comparing the conditional distributions of the original data and the generated composite data. For instance, the similarity may have a value between 0 and 1, but is not limited thereto.

The processor 230 may determine whether the composite data is generated similarly to data items belonging to a category in the original data based on the calculated similarity. In addition, the calculated similarity may be fed back to the tabular data generator as a loss, and a learning weight value may be copied and transferred.

In one embodiment, as shown in FIG. 12, subsequent to learning, by a model, the contents of materials and the distribution diagram of data for each property in each formulation of DATA 1, the processor 230 may sample composite data having a distribution similar to DATA 1, such as DATA 1-1, DATA 1-2, and DATA 1-3. Meanwhile, in composite formulation data augmentation, existing input data and noise vectors may be input to a trained generator. Composite data similar to existing data may be generated based on the output of the trained generator, and a new dataset including the existing data and the composite data may be built to augment the data.

Referring to FIGS. 13 to 15, prior to generating composite formulation data, the processor 230 may perform preprocessing to facilitate the learning of the model. The preprocessing process may be carried out to allow use in the form of a single table since the formulation information and property information of materials are respectively built as separate tables in the database built in step S100. The processor 230 may select data items with the same material types and experimental properties and then preprocess the corresponding data items.

In one embodiment, in step S200, the generating of the composite formulation data may further include selecting data items with the same types of materials and experimental properties from the formulation database, and performing preprocessing on the selected data items. Here, as shown in FIG. 13, in order to distinguish each formulation information, data items included in a formulation database may be converted into the form of a row in which the type of each material is a column based on the set identifier (id) and the content of the material is a value. Similar to a formulation table, data items in a property table may also be converted into the form of a row in which the type of each test property is a column and the property value is a value based on the identifier (id) as shown in FIG. 14. Then, the data items that have been converted into the form of a row in the formulation database and the property database may be merged based on each identifier (id) and finally converted into the form of a data frame as shown in FIG. 14.

Referring to FIG. 16, data that has been normalized is used as learning data to generate composite formulation data. Here, a composite formulation data generation model may learn the distribution diagram of input data to generate data with a distribution diagram similar to the input data. In addition, as shown in FIG. 16, distribution diagrams of data input to the composite formulation data generation model and composite data generated therefrom are illustrated. An example in FIG. 16 is a result of classifying about 82 formulations of mPPO, which is a type of compound, into 57 train sets and then sampling 1000 composite data, and shows distribution diagrams of data input into the composite formulation data generation model and distribution diagrams of 1000 composite data sampled by the composite formulation data generation model.

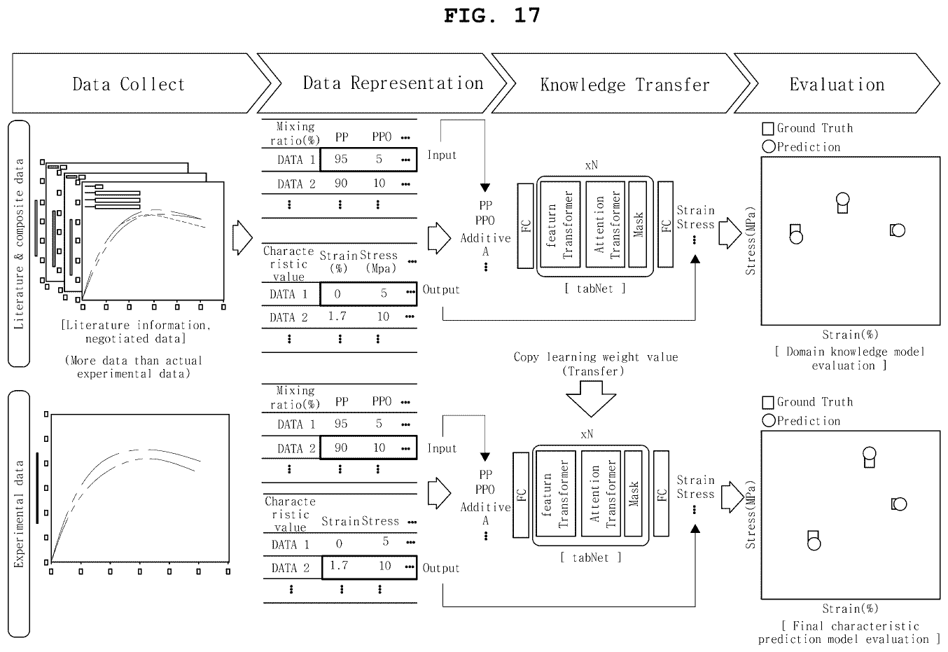

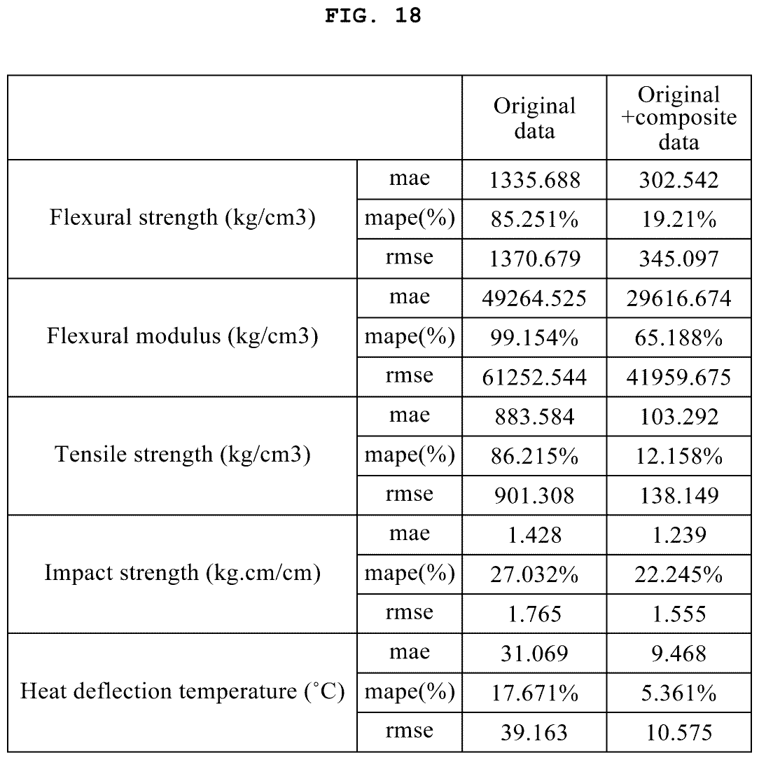

FIG. 17 is a diagram for explaining a formulation property prediction process according to an exemplary embodiment of the present disclosure, and FIG. 18 is a diagram showing examples of data generated by a formulation property prediction model according to an exemplary embodiment of the present disclosure.

Referring to FIG. 17, in an exemplary embodiment, a formulation property prediction model may infer a property change depending on a formulation ratio because a change in property is related to proportions of formulation materials. The formulation property prediction model may individually carry out training for each property. A formulation property prediction model that has been trained may receive formulation information of a compound to predict the property of the corresponding compound. Here, indicators for evaluating the prediction performance of the model may be used, for example, MAE, MAPE, f1-score, and the like.

In one embodiment, step S300 may include collecting learning data including preprocessed original data and the composite formulation data, selecting a property value to be predicted by the formulation property prediction model, and then defining a representation form of data, storing a weight value of a model for each property that has been trained through knowledge transfer and carrying out performance evaluation on untrained data, and evaluating a prediction result for each predicted property.

In the collecting of learning data, the collection of learning data is performed by selecting data items with similar experimental properties and types of materials, and then passing through the same process as the preprocessing process that has been carried out for an input to the composite data generation model in step S200. Then, learning data for the model is collected by merging the original data that has passed through the preprocessing process and the composite formulation data generated in step S200.

In the defining of the representation form of data, for data representation, subsequent to selecting a property value to be predicted by the model, the representation form of data is defined. For example, when predicting a property such as a flexural strength, materials that make up a formulation and the content information items of the materials are classified as input values of the model. Here, the contents of materials, which are input features of the model, are embedded to facilitate selection of important features and knowledge transfer.

A weight value of a model for each property that has been trained through knowledge transfer may be stored, and performance evaluation on untrained data may be carried out by storing a weight value of a model for each property that has been learned through knowledge transfer in the carrying out of performance evaluation on untrained data.

In an exemplary embodiment with respect to the evaluating of a prediction result for each predicted property, the evaluating of the prediction result for each predicted property may include evaluating the prediction result using a first performance evaluation index for a first predicted property corresponding to a continuous variable, and evaluating the prediction result using a second performance evaluation index different from the first performance evaluation index for a first predicted property corresponding to a categorical variable. For example, a prediction result for each property is evaluated by using an index such as MAE and MAPE when the predicted property is a continuous variable such as a flexural strength, and the prediction result for each property is evaluated by using f1-score for a categorical variable such as a flame retardancy.

An execution result of the property prediction model may be illustrated as shown in FIG. 18. The corresponding example shows a case where composite data generated in step S200 is used and a case where the composite data is not used. Similar to an example of a composite data generation model, the formulation data of mPPO was used. A total of 82 data are classified into 57 training sets. FIG. 18 shows a case where the training of the property prediction model is carried out for only 57 data items subsequent to classification, and a result of sampling 1,000 composite data items and then carrying out prediction on 25 test sets using a total of 1,057 data items to train the property prediction model.

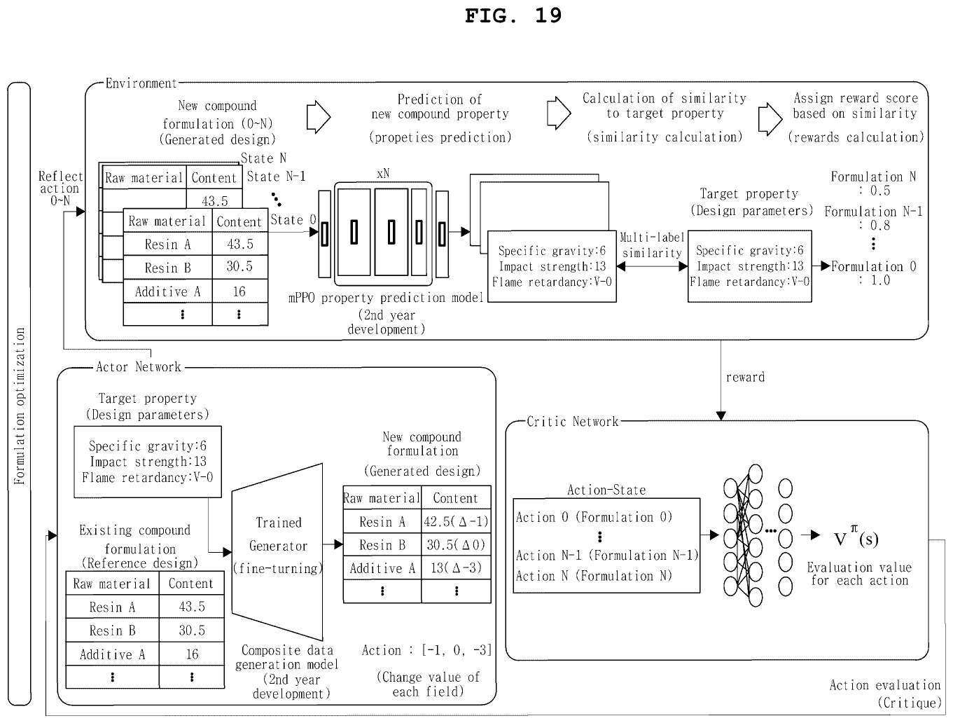

FIG. 19 is a diagram for explaining a formulation optimization process according to an exemplary embodiment of the present disclosure.

Referring to FIG. 19, in an exemplary embodiment, a formulation optimization model allows reverse engineering to accelerate formulation optimization according to a target property.

In one embodiment, a process of formulation optimization may be carried out as shown in FIG. 19. The formulation optimization model may include an actor network that generates various new compounds using a deep reinforcement learning-based model, and a critic network that evaluates how much a previously generated new formulation has improved based on a reward score of a newly generated formulation. Step S400 may utilize an actor-critic based reinforcement learning model to generate a new compound formulation suitable for a target property from an existing compound formulation.

In one embodiment, an actor-critic based reinforcement learning method for optimizing a formulation may have the following features.

First, in an actor network, a trained generator may generate a variety of new compound formulations by receiving an existing compound formulation and a target property as an input.

Second, in an interactive environment, a property prediction model according to an exemplary embodiment may be used to measure a property value of a new compound formulation generated in an actor network. A property prediction model according to an exemplary embodiment may receive a new compound formulation, predict a property value of the new compound formulation, calculate a similarity to the target property, and assign a reward score based on the similarity through the calculation of the similarity. A similarity score is assigned as a real number between 0 and 1, and since the goal of the formulation optimization model is to optimize a formulation close to a target property, the similarity score may be closer to 1 as the similarity to the target property increases.

Third, a similarity-based reward score may be delivered to a critic network. In a critic network, an expected value of how close a new formulation changed compared to an existing formulation has improved to a target property may be estimated and fed back to an actor network. The actor network may be a module that generates a new compound formulation by receiving information on the existing compound formulation and target property. An actor plays a role in determining an action in a given state, which may be referred to as a policy, and in the present disclosure, the policy of an actor model may be adjusted by changing a content value of the formulation of each material to positive (+), negative (−), or ‘no change’. A weight value of a composite formulation data generation model that has been previously trained in step S200 may be transfer learned using a baseline model of the actor network. Through reinforcement learning, a weight value of the actor network may be fine-tuned to learn new compound formulation data suitable for the target property.

In one embodiment, step S400 may include generating, in an actor network, third data for a new compound formulation based on first data for a target property and second data for an existing compound formulation, assigning, in an interactive environment, a reward score based on a similarity between a property value of the new compound formulation of the third data and the target property, and feeding back, in a critic network, an expected value of how close the new formulation changed compared to an existing formulation has improved to the target property based on the reward score to the actor network.

In an exemplary embodiment with respect to step S400, the assigning of a similarity-based reward score may include predicting the property value of the new compound formulation using the formulation property prediction model, calculating a similarity between the predicted property value and the target property, and calculating a reward score corresponding to the similarity.

The present disclosure has been described with reference to an embodiment illustrated in the drawings, but the embodiment is merely illustrative, and is not limited to the above-described embodiments, and it should be appreciated by those skilled in the art that various modifications and other embodiments equivalent thereto can be made therefrom. Therefore, the true technical protective scope of the present disclosure should be determined only by the technical concept of the appended claims.

### DESCRIPTION OF SYMBOLS

100: System
110: User terminal
120: Electronic device
130: Network

What is claimed is:

1. An operating method, performed by one or more processors of an electronic device, the operating method comprising:
extracting data from a document to build a formulation database;
generating composite formulation data based on the formulation database;
predicting, using a formulation property prediction model that infers a property change according to the composition of a formulation, the property of a compound from the formulation information of the compound based on the composite formulation data; and
optimizing a formulation using a formulation optimization model that generates a new compound formulation suitable for a target property in a compound formulation,
wherein the building of the formulation database comprises:
extracting a paragraph related to a formulation from text included in the document; and
extracting data related to the formulation from a table included in the document, and
wherein the extracting of data related to the formulation comprises:
checking a row having composition information or property information in a table included in at least one of an HTML document and an XML document;
sorting example numbers assigned to classify experimental examples in a table head, the composition information, and the property information, respectively, to generate a composition data set and a property data set;
extracting a pair of composition information and property information whose example numbers match from the composition data set and the property data set;
extracting information including the name, unit, and amount of a raw material from the pair of the composition information and the property information;
extracting information including the name, value, unit, property experimental method, and property experimental condition of a property from the pair of the composition information and the property information; and
automatically adding information including the name, unit, and amount of the raw material and information including the name, value, unit, property experimental method, and property experimental condition of the property to an existing database.

2. The operating method of claim 1, wherein the extracting of a paragraph related to the formulation comprises:
extracting text segments in the document;
predicting a class related to the formulation for the extracted text segments using an object name recognition model; and
storing a paragraph including a sentence including information including at least one of the composition and property of the formulation.

3. The operating method of claim 1, wherein the extracting of data related to the formulation comprises:
checking the locations of tables in the document using a pre-trained table-transformer model;
setting a window of a preset size at the checked locations of the tables;
extracting contents of a table included in the window;
selecting a table including a content including a keyword related to the formulation from the extracted contents using a Tesseract OCR model; and
storing the selected table.

4. The operating method of claim 3, wherein the extracting of data related to the formulation further comprises:
converting selected table images into an HTML format using a table structure recognition model; and
converting and storing a table to fit the format of the existing database using an HTML parser.

5. The operating method of claim 1, wherein the generating of the composite formulation data comprises:
generating composite formulation data from original data using a tabular data generator;
comparing a conditional distribution between the original data and the composite formulation data using a discriminator to output a similarity; and
sampling composite data based on the similarity.

6. The operating method of claim 1, wherein the predicting of the property of the compound comprises:
collecting learning data including original data and the composite formulation data;
storing a weight value of a model for each property that has been trained through knowledge transfer, and carrying out performance evaluation on untrained data; and
evaluating a prediction result for each predicted property.

7. The operating method of claim 1, wherein the optimizing of the formulation comprises:
generating, in an actor network, third data for a new compound formulation based on first data for a target property and second data for an existing compound formulation;
assigning a reward score based on a similarity between a property value of the new compound formulation of the third data and the target property; and
feeding back, in a critic network, an expected value of how close the new formulation changed compared to an existing formulation has improved to the target property based on the reward score to the actor network.

8. An electronic device that performs the method of claim 1, the electronic device comprising:
a memory that stores one or more instructions; and
a processor that executes the one or more instructions stored in the memory,
wherein the processor executes the one or more instructions.
